(19) United States
(12) Patent Application Publication
(10) Pub. No.: US 2005/0041817 A1
(43) Pub. Date: Feb. 24, 2005

(54) Ear cover with sound receiving element

(76) Inventor: Adelbert Willem Bronkhorst, Baarn (NL)

Correspondence Address: YOUNG & THOMPSON, 745 SOUTH 23RD STREET, 2ND FLOOR, ARLINGTON, VA, 22202, US

(21) Appl. No.: 10/496,392

(22) PCT Filed: Nov. 25, 2002
(86) PCT No.: PCT/NL02/00763

(30) Foreign Application Priority Data
Nov. 23, 2001 (NL) 1019428

(51) Int. Cl. H04R 1/10; A61F 11/06

(52) U.S. Cl. 381/72; 381/74; 381/384

(57) ABSTRACT

An ear cover such as a pair of ear caps or a helmet is provided with a spatial array of several sound pickup elements on an outer wall of the ear cover. Within the ear cover sits a sound reproduction element. A signal processing device composes an output signal for reproduction by the sound reproduction element from a composite of respective input signals picked up by different elements of the array, of the sound from the environment. The composition is preferably carried out such that it approximates as best as possible the directional dependence of the transfer of sound to the ear canal, such as it would occur in the absence of the ear cover.

The invention relates to an ear cover which is provided with a sound pickup element for picking up sound from an environment of the ear cover and a sound reproduction element for reproducing the sound within the ear cover adjacent an ear that is screened off by the ear cover.

Ear covers, specifically hearing protectors, are generally designed in the form of a cap which is placed over the ear. Besides caps that are placed over individual ears, helmets or caps placed over the entire head for protection also have an ear covering effect. The ear covering prevents sound from the environment reaching the ears of a user undamped. Simple ear covers, which only give passive sound damping, have the disadvantage that the user cannot respond properly to environmental sounds because he or she is more or less being shut out from them. This disadvantage can arise also with helmets that are not provided with hearing protection but do screen off the ears.

To control this problem, it is known to include a sound pickup element and a sound reproduction element in hearing protectors, to pass environmental sound to the ear of the user. The sound pickup element, for instance a microphone, is on the outside of the hearing protector and converts the environmental sound to an electrical signal. The sound reproduction element is on the inside of the hearing protector and converts the electrical signal to sound which is passed on to an ear of the user. This can be accompanied with attenuation or amplification of the sound to an acceptable level, or with filtering out of detrimental or objectionable sound components, such as extremely loud sounds or sounds in a particular frequency range.

It is also known to use a separate sound pickup element for each ear, so that the user can perceive the environmental sound in a stereophonic directionally sensitive manner. Application of a sound pickup element and a sound reproduction element in helmets that protect against mechanical hazards, such as military helmets and construction helmets, however, is not known.

Despite the use of sound pickups, the known ear covers still prove to have a clear effect on hearing. It turns out that the positions of sound sources in the environment of the user can be determined less well than with uncovered ears.

It is one object of the invention to provide an ear cover which causes less appreciable deterioration of the spatial hearing, and which enables the user to determine the directions of environmental sounds in a more normal manner.

The invention provides an ear cover provided with
a spatial array of several sound pickup elements in or on a wall of the ear cover for picking up sound from an environment outside the ear cover;
a sound reproduction element for reproducing the sound within the ear cover adjacent an ear that is covered by the ear cover; and
a signal processing device for composing an output signal for reproduction by the sound reproduction element from a composite of respective input signals which have been picked up by different elements of the array, of the sound from the environment.

The invention utilizes the insight that the reason for the deterioration of the spatial hearing is that, with open ears, specific direction-dependent acoustic effects arise, defined by the shape of the body (in particular the head and the auricles), which are not reproduced by the known sound relay systems.

A more natural directional dependence is rendered possible by composing the reproduced sound from a combination of the different input signals which have been picked up by an array of sound pickup elements on the ear cover.

The transfer functions which determine the contribution of the different input signals to the reproduced sound are preferably selected such that they realize a direction-dependence of transfer of environmental sound to sound in the ear canal that approximates the natural acoustic transfer function of the open ears as much as possible, albeit possibly amplified or attenuated and optionally whilst filtering out sound components considered detrimental or objectionable. For a number of applications, however, an unnatural directional dependence is useful, for instance if the acoustic effects of a space in which the wearer finds himself must be compensated or, for instance, if an adjustment is necessary of the angular range over which the strongest sensitivity arises, or in which only sounds from particular ranges of sound directions lead to natural transfer, and so forth.

In one embodiment, the transfer functions are so arranged that a transfer of sound from a direction from a range of twenty-five to seventy degrees to the side of the forward direction from the head is maximal, compared with directions having closer angles with respect to the forward direction.

Without the ear covering, the shape of the auricles and the presence of the head normally provide for a maximum sensitivity to sound from such a direction. This is restored by the transfer functions when using the ear cover. The precise direction in which the maximum occurs varies from person to person. The direction of the maximum is preferably virtually in the horizontal plane through the ears. Also, the precise direction depends on the frequency range over which the strength of the transfer is averagely determined. For this, preferably a range of low and medium frequencies is used (for instance from 0 to 5 kHz).

The invention is preferably used for coverings of both ears, with each ear having an own array of sound pickup elements. Thus, both ears are protected. The arrays are preferably situated as close as possible to the respective ears for which they pick up the sound.

According as the array(s) contain more sound pickup elements, an increasingly more natural sound reproduction will become possible. With two sound pickup elements per ear, however, already a clear improvement is obtained. The elements for one ear are then preferably placed such that they differ in position viewed along an axis perpendicular to the vertical plane passing through both ears, and still more preferably in a horizontal plane through the ear. The distance between the pickup elements can be chosen freely, but preferably so that an optimum natural effect is rendered possible. With two sound pickup elements, a mutual distance of two centimeters proves to be properly satisfactory.

The desired transfer functions can for instance be determined experimentally, based on a comparison of sound measurements performed with an array of sound pickup elements, with sound measurements in the ear canal of a test subject or artificial head. For this purpose, also simulations or theoretical calculations can be used. The desired transfer functions can, if desired, be determined specifically for a particular user, taking his or her build into account, but more generally approximated transfer functions are also possible.

These and other objects and advantages of the ear protector according to the invention will be further described with reference to the following figures.

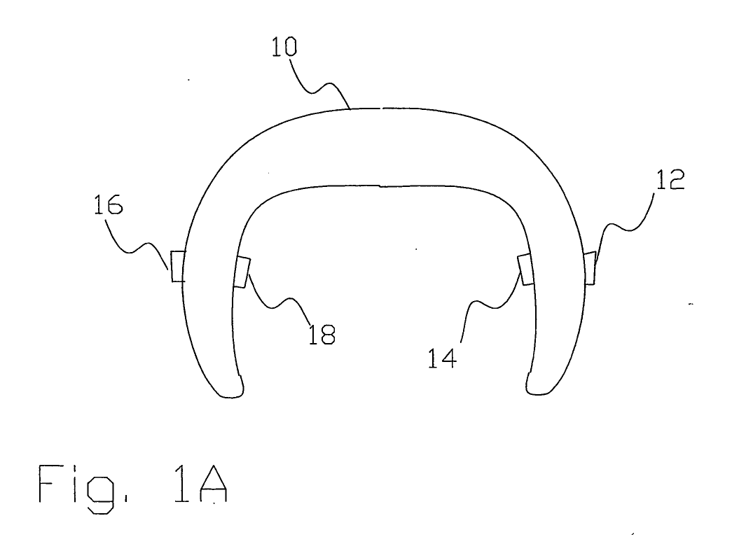

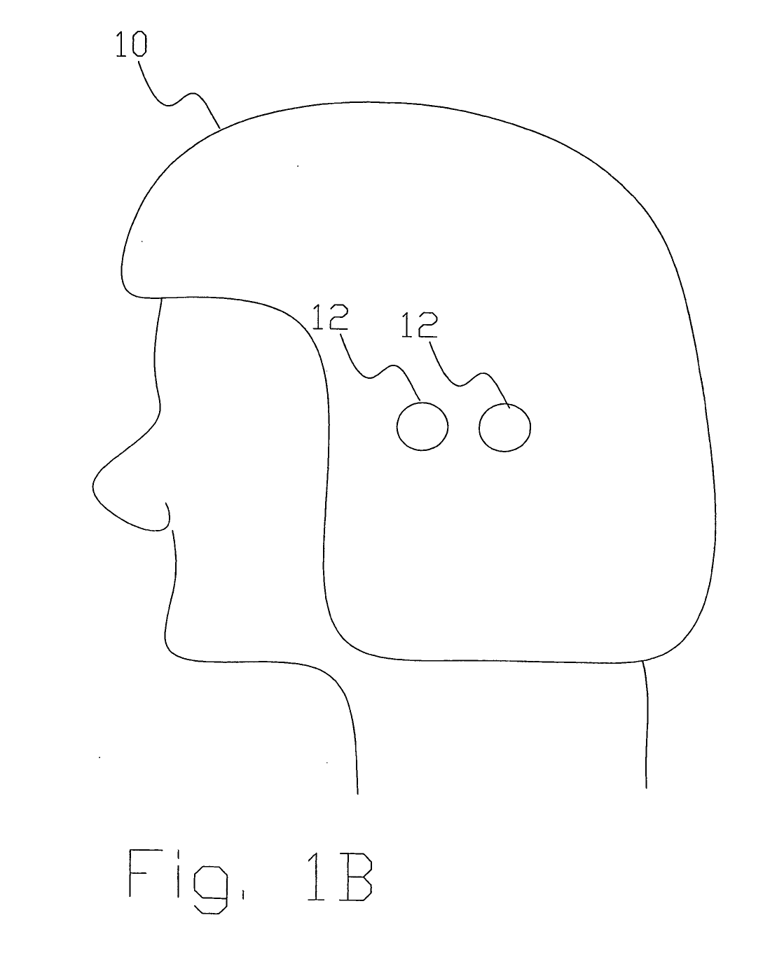

FIGS. 1A-B show an ear cover

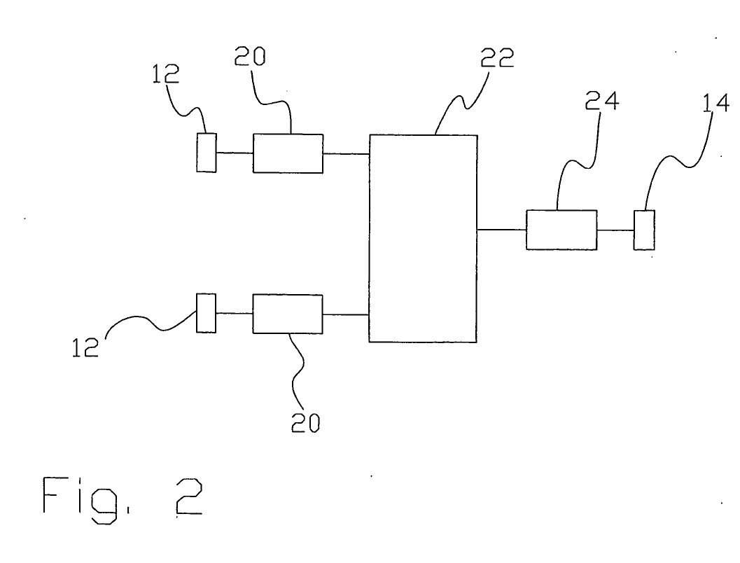

FIG. 2 shows a signal processing circuit

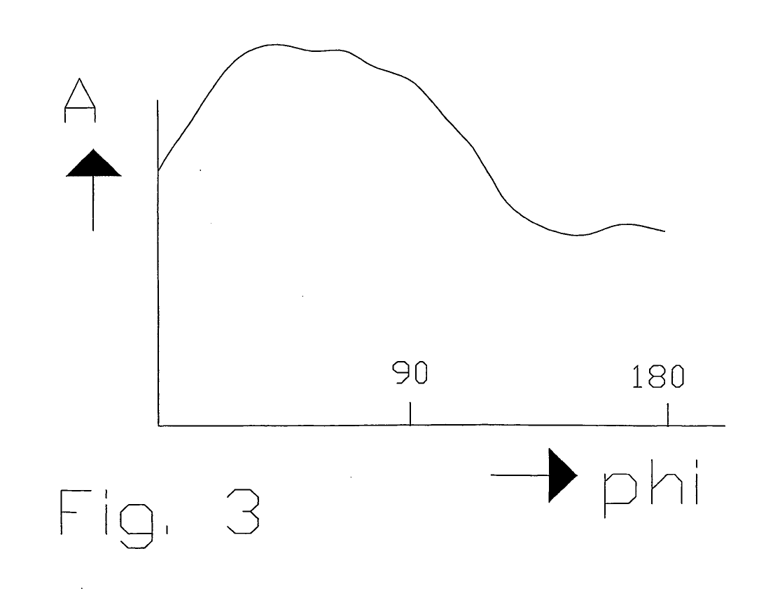

FIG. 3 shows a sensitivity characteristic

FIGS. 1A-B show, in front and side elevation, an ear cover in the form of a helmet 10, with a first array of sound pickup elements 12 on the outside of the helmet 10 and a first sound reproduction element 14 on the inside of the helmet 10. Further shown are a second array of sound pickup elements 16 (only one is visible in front view) on the outside of the helmet 10 and a second sound reproduction element 18 on the inside of the helmet 10. The sound reproduction elements 14, 18 are each connected via their own signal processing circuit (not shown in FIG. 1) with several of the sound pickup elements 12, 16.

FIG. 2 shows sound pickup elements 12, sound reproducing element 14 and the signal processing circuit for forming the signal for the sound reproducing element 14 for one of the ears. The signal processing circuit includes A/D converters 20, a digital signal processor 22 and a D/A converter 24. The digital signal processor 22 is coupled via A/D converters to the different sound pickup elements and via D/A converter 24 to the sound reproducing element 14. In principle, for each of the ears, a circuit as shown in FIG. 2 can be included in the helmet 10, but if desired a greater or lesser part of the signal processing circuit can be shared for the two ears.

Although two pickups 12 per ear are shown, it will be clear that the invention is not limited to this number. More pickups 12 can be used per ear, each coupled, for instance via its own A/D converter 20, to the signal processor 22. Nor is the invention limited to the positioning of the pickups 12 as shown. Preferably, the pickups 12 are arranged as close to the ear as possible. Also, the distance between the pickups 12 is merely illustrative. A distance of more than 1 centimeter (for instance 2 centimeters) between the pickups 12 for one ear has proved useful. Preferably, however, the pickups 12 are not all placed in the same vertical plane (horizontal and vertical here refer to these directions when wearing the ear cover, with the head held upright). Thus, it is possible to discriminate between sound that approaches the head from the front and from the back. In the case of two pickups 12, the pickups are, for instance, preferably placed behind each other in the same horizontal plane.

In operation, a user wears the helmet on the head. The sound pickup elements 12, 16 convert environmental sound to electrical signals, which are converted by A/D converters 20 to sampled digital signals. Signal processor 22 calculates from these digital signals a composite digital signal. Signal processor 22 passes this composite signal to D/A converter 24, which converts it and drives a sound reproducing element 14 with it.

The signal processor 22 calculates the composite signals Oj(t) (wherein j is an index for the different sound reproducing elements 14, 18) essentially with a linear operation, so that the output signal of signal processor 22 Oj(t) at time t is a sum of signals Si(t′) of the different A/D converters 20 (labeled i=1, 2 . . . ) at times t′ (<t), weighted with filter coefficients h(i,t), for instance according to the relation:
Oj(t)=sum over i and t′ h(i,t-t′) Si(t′)
(This is only an example: O(t) could also depend on O(t′), with (t′<t), or on intermediate signals formed from Si). The filter coefficients h(i,t) can be positive, negative or zero. They describe the transfer functions of the signal Si of the pickups 12, 16 to the reproduction elements 14, 18 and generally differ for the signals from different A/D converters 20. In principle, the signal Oj(t) for an ear “j” is composed only from the signals Si of the pickups 12, 16 adjacent the respective ear “j”, but without deviating from the invention, use can also be made of signals Si picked up at the other ear.

The filter coefficients h(i,t) are preferably chosen such that the sound from sound reproducing element 14 that reaches the eardrum of the ear on that side corresponds as much as possible with the sound that would reach the ear drum in the complete absence of the helmet, except that the sound is possibly amplified or attenuated (optionally controlled such that a particular average sound level is realized) and that detrimental or objectionable elements (for instance extremely loud sounds, or sounds in particular frequency ranges) are eliminated or attenuated.

FIG. 3 shows a natural sensitivity characteristic for sound of low to medium frequencies (frequencies in a range of 0 to 5 kHz) as a function of the angle “phi” at which the sound approaches an ear in a horizontal plane through the ear. The angle 0 corresponds to the forward direction (the direction perpendicular to a vertical plane through the ears). The sensitivity “A” is represented with a logarithm ratio between the sound strength of the sound source and the strength of the resulting sound in the ear canal. The sensitivity varies over approximately seven decibels. Visible is that this characteristic has a maximum for forty degrees off the forward direction, on the side of the head where the ear is situated. This is, for one thing, a result of a shape of the head and the auricle. The angle at which the maximum occurs can differ from one person to another, but is mostly in a range of twenty-five to seventy degrees.

A directional sensitivity which has a maximum in such a direction can be realized with the pickups 12, for instance by adding the signals of the different pickups in the signal processor 22 with a mutually different delay, so that the delay in the signal processor cancels out the mutual delay with which the sound from the direction involved reaches the different pickups 12. Further, the signals can be given different weights in the addition. In addition, the signals can be filtered, for instance to suppress higher frequencies, for instance frequencies which for other directions could lead to maxima in the sensitivity characteristic other than the maximum of FIG. 3.

Preferably, however, the filter coefficients are optimized for obtaining a desired directional dependence. This can be done in various ways. In one example, the coefficients are determined with the aid of an artificial head. The artificial head has the shape of a head and comprises an ear canal, in which a sound pickup is included. The artificial head is placed in a space without the helmet 10. Next, in the space, test signals are generated from different directions and the resultant signals at the output of the sound pickups in the two ear canals are measured. Next, this procedure is performed once again, but with the helmet 10 placed on the artificial head, and utilizing the sound pickup elements 12, 16, on the helmet instead of the sound pickups in the ear canals. From the measurements, the acoustic transfer functions of the sound pickups in the ear canal and on the helmet are calculated. The filter coefficients are chosen such that the differences between the transfer functions of the sound pickup in the ear canal and those of the array of sound pickups on the helmet, averaged over frequency and sound direction, are minimal.

In one embodiment, one determines the phase (with respect to the phase of the sound source) and amplitude of the sound received in the ear without hearing protector, which is expressed in a complex factor A(f,x) as a function of the frequency, f, and the direction, x, of the sound. Determined next are the phase and amplitude of the sound received outside the helmet as a complex factor Bi(f,x) for the different pickups, labeled “i”. Instead of the phase and amplitude for a specific frequency, it is also possible to take for A, B the transfer factors between source and pickup of the sound with a spectrum in a frequency band around f, or to utilize a broadband source, from which factors A and B are determined afterwards. The transfer functions Hi(f) to be chosen (Fourier transform of the filter coefficients h(i,t)) lead to a sound whose phase and amplitude are determined by
sum over i Hi(f,x)B(f,x)
at the point where the signal A was picked up. Now, a deviation measure Q is defined:
Q=integral of sum over f and x of w(f,x)|A(f,x)−sum Hi(f,x)Bi(f,x)|²
(wherein w(f,x) is a weighting function which weighs the different frequencies f and directions x). If desired, the weighting function w(f,x) can be taken constant over all directions and frequencies within the range of human hearing. In an embodiment, directions with different angles relative to a vertical axis through the head are given different weight, so that directions in a band around the horizontal plane are given maximum weight. Also, phase differences and amplitude differences between A(f,x) and the sum Hi(f,x),Bi(f,x) can be given different weights: preferably, the phase difference is given less weight than the amplitude difference. This has been found to counteract the formation of artifacts.

The filter coefficients Hi are selected such that they minimize Q. Obviously, this is just an embodiment. The transfer functions H can also be calculated in a different manner, for instance by making use of computer simulations or model calculations. Nor is it essential to calculate in terms of a function of the frequency f and with factors A and B: the same calculation can be performed with time-dependent transfer functions. If the sound reproducing elements 14, 18 do not have a flat transfer characteristic, it may be necessary to implement a correction for this, in the form of a filter that has the inverse of this transfer characteristic. This filter can be realized separately, or it can be integrated into the filters which have been linked up after the sound pickup elements.

An experiment as described above can also be carried out with the head of a concrete user, in whose ear canals miniature sound pickups are arranged.

The measurements with artificial heads and in users enable different embodiments, which can be realized by loading the filters in the digital signal processor with different sets of coefficients. In one embodiment, intended for the most demanding applications, the transfer function of the array of sound pickups including digital filters and sound reproduction element will correspond as accurately as possible with the transfer function of the open ears of the individual user. In another embodiment, the user can choose from a number of pre-programmed transfer functions, with which an adequate approximation of one's own transfer function can be realized. In the simplest embodiment, there is only one transfer function incorporated, for instance based on an artificial head or a representative test subject.

1. An ear cover provided with
a spatial array of several sound pickup elements in or on a wall of the ear cover for picking up sound from an environment outside the ear cover;—a sound reproduction element for reproducing the sound within the ear cover adjacent an ear that is covered by the ear cover; and
a signal processing device for composing an output signal for reproduction by the sound reproduction element from a composite of respective input signals which have been picked up by different elements of the array, of the sound from the environment.

2. An ear cover according to claim 1, wherein the signal processing device has each of the input signals contribute to the output signal with an own transfer function; wherein there is a deviation between a direction-dependence of transfer of the sound from the environment to sound within the ear cover and a natural acoustic transfer of the sound from the environment to the ear canal that would arise in the absence-of the ear cover, and wherein the transfer functions used are set such that the deviation under a set of possible deviations that would occur for different possible transfer functions is substantially minimal.

3. An ear cover according to claim 1, wherein the signal processing device has each of the input signals contribute to the output signal for reproduction with an own transfer function, and wherein the transfer functions are set such that a strength of the transfer of the sound from the environment to sound within the ear cover when wearing the ear cover as a function of an angle between a head on which the ear is located and a direction from which the sound comes is maximal for sound from a direction which, viewed from the covered ear, points away from the head, being in an angular range of twenty-five to seventy degrees with respect to a forward axis of the head.

4. An ear cover according to claim 1, provided with covering for both ears; wherein there is provided for each ear an own spatial array of sound pickup elements and sound reproduction element; wherein the signal processing device is arranged for composing an own output signal for reproduction by the sound reproduction elements for each ear; wherein the own output signal is composed from a composite of at least the different input signals which have been picked up by elements of the array of sound pickup elements for the respective ear.

5. An ear cover according to claim 4, wherein the signal processing device has each of the input signals contribute to its own output signals with their own transfer function, and wherein the transfer functions are set such that a strength of the transfer of the sound from the environment to sound within the ear cover when wearing the ear cover as a function of an angle with a head on which the ears are situated is maximal for each ear for sound from a direction which, viewed from the respective ear, points away from the head, lying in an angular range of twenty-five to seventy degrees with respect to a forward axis of the head.

6. An ear cover according to claim 1, designed as a helmet which covers at least a part of the head that includes the two ears, wherein the sound pickup elements are included on or in an outer wall of the helmet, and the sound reproduction element is included on or in an inner wall of the helmet adjacent at least one of the ears.

7. An ear cover according to claim 6, wherein the inner wall of the helmet is provided with sound reproduction elements for both ears.

8. A method for manufacturing a hearing protector according to claim 1, which method includes the steps of
determining sound signal values of sounds that would reach an ear canal without the ear cover from different directions;
calculating filter coefficients for the transfer functions with which each of the input signals contributes to the output signal with an own transfer function, so that the filter coefficients minimize a calculated difference between the composite signal and the determined sound signal, weighted over a number of directions;
programming the signal processing device with the calculated filter coefficients.

9. A method for manufacturing a hearing protector according to claim 8, which method includes the steps of
setting up sound pickup elements in said spatial array relative to a head,
presenting sound to the sound pickup elements from different spatial directions and measuring resulting input signals;
setting up a further sound pickup element in an ear canal of the head;
presenting sound to the further sound pickup element from the different spatial directions and measuring a further signal on the further sound pickup element;
calculating filter coefficients, so that a difference between the composite sound and the further signal, averaged over the different directions, is jointly minimized.
